(19) United States
(12) Patent Application Publication
(10) Pub. No.: US 2025/0352386 A1
(43) Pub. Date: Nov. 20, 2025

(54) Insertable Anti-Leaking Stoma or Ostomy Device

(71) Applicant: Paige Cisna, Granger, IN (US)

(72) Inventor: Paige Cisna, Granger, IN (US)

(21) Appl. No.: 18/665,409

(22) Filed: May 15, 2024

(51) Int. Cl. A61F 5/445

(52) U.S. Cl. CPC A61F 5/445

(57) ABSTRACT

An insertable anti-leaking ostomy or stoma device and a method of inserting it. The device includes a channel or tube having a first end, a first inflatable balloon on the first end of the tube, and a second inflatable balloon on the second end of the tube. The device is inserted into the stoma by first being rolled up and inserted into the plastic base of an applicator, sterile lubricant being applied to the applicator, the plastic base with soft silicone tip being inserted into the stoma, a plunger of the applicator being used to push the rolled up first end into the stoma in which the ostomy drains, the plastic base and applicator being removed from the device and patient, and then inflating the first balloon via a balloon port using sterile saline solution or water.

### CROSS-REFERENCE TO RELATED APPLICATIONS

This application has no related applications.

### BACKGROUND OF THE INVENTION

A stoma, also called an ostomy, is an artificial opening in an organ of the body, created during an operation such as colostomy, ileostomy, or urostomy. A common complication with an ostomy pouching system, used to catch effluent exiting through a stoma, is leaking. This leaking often results in skin breakdown, wounds, overuse of supplies, and overall decreased quality of life.

In particular, an ileostomy or urostomy generally discharges a loose or liquid type of effluent. The present invention is directed toward an anti leaking device for the stoma after an ileostomy has been performed, but can be used for any stoma for which it fits the criteria for use.

### SUMMARY OF THE INVENTION

A device used to direct or funnel output from a stoma into the ostomy bag or pouch, to prevent leaking, pouch breakdown, and skin disturbances. The device includes a flexible silicone catheter which is slightly structured, as well as a surrounding ring shaped balloon. The deflated balloon and attached end of the catheter are first inserted into the stoma and then inflated with sterile water or saline via an external port, similar to a Foley catheter, such that the channel is held open by the balloon and the device is held in place under the fascial layer by the ring shaped balloon. The device may also include an outside inflatable ring shaped balloon, extending approximately ¾ of the length of the inside inflatable ring shaped balloon, to maintain the openness of the channel and help funnel liquid into the pouch. The balloon or balloons can be deflated for removal or replacement of the device, by using a syringe to remove the sterile water or saline through the external port.

The insertion of the ostomy device is accomplished via an applicator, similar to a tampon applicator, which includes a plastic base which the device is initially within, a soft silicone tip, and a plunger used to push the device out of the plastic base. The ostomy device can be made in different sizes, with individual length depending on the amount of subcutaneous adipose tissue, as well as the diameter of the balloons and channel depending upon the type of ostomy. Similarly, the applicator may vary in dimensions according to the specifications of the device.

### BRIEF DESCRIPTION OF DRAWINGS

FIG. 1: the ostomy device, shown in cross section after insertion into the body and inflation of the first balloon.

FIG. 2: An alternative embodiment of the ostomy device, including a second balloon, shown with an attached ostomy pouch or bag and after insertion into the body and inflation of both balloons.

FIG. 3: The ostomy device of FIG. 1, shown inside the applicator prior to insertion into the body.

### DETAILED DESCRIPTION OF THE INVENTION

In the following description, numerous specific details are set forth to clearly describe the embodiments disclosed herein. However, one skilled in the art will understand that some well known features have not been described in detail so as not to obscure the invention.

While illustrative embodiments of the invention have been shown and described, variations and alternative embodiments will occur to those skilled in the art. Such variations and alternative embodiments can be made without departing from the scope of the invention as defined in the claims.

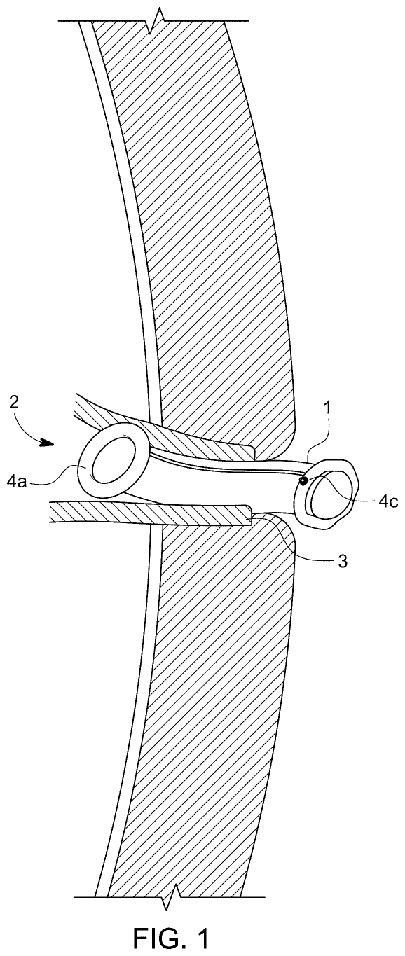

As illustrated in FIG. 1, the ostomy device (1), illustrated relative to the peritoneal cavity (2), also known as the space within the abdomen containing intenstines, stomach and liver; the stoma (3); and the subcutaneous fat. As illustrated in FIG. 1, the end of the channel of the device inside the lumen of the intestines under the fascial layer has a first balloon (4a), which is inflated after insertion into the stoma via the balloon valve (4c) to ensure the device stays in place without leaking. The balloon valve permits the use of sterile water or saline to inflate the balloon. Although the only illustrated embodiment has both first and second balloons, the disclosure of the invention is explicitly intended to encompass an alternative embodiment in which there is only one balloon on the inside, without a second ring shaped balloon on the outside.

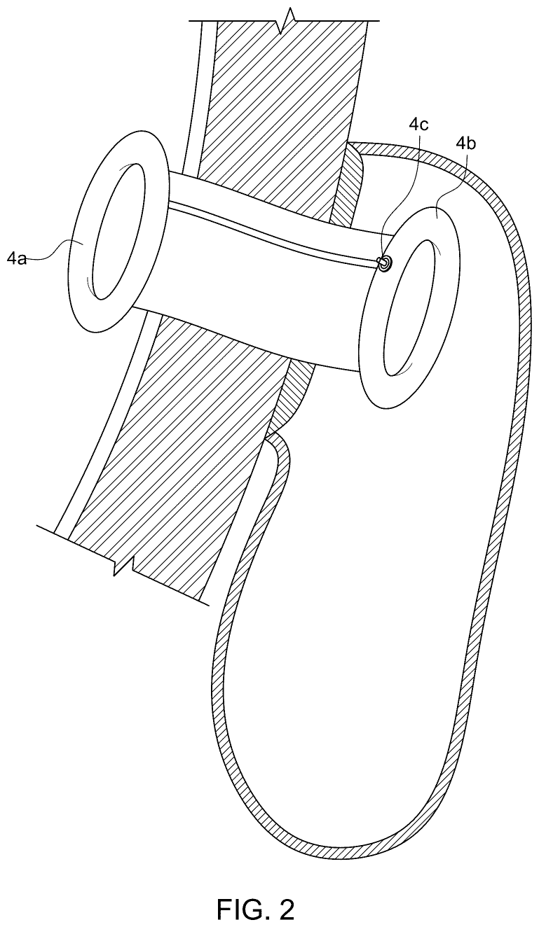

As illustrated in FIG. 2, the end of the device which is outside the body is configured to drain into to an ostomy pouch adhered to the body. The end of the channel of the device outside the body has a second balloon (4b), which can also be inflated via a balloon valve (4c), which is separate from and additional to the balloon valve used to inflate the first balloon (4a). This second balloon serves to funnel and direct effluent through the channel and into the ostomy pouch more efficiently, thereby reducing the likelihood of leaks or upflow of effluent from the pouch.

Although not separately illustrated, the second balloon 4b may be inflated via the same balloon valve 4c as is used to inflate the first balloon. In order to omit the second balloon valve, a channel fluidly connecting the first and second ring shaped balloons must be present.

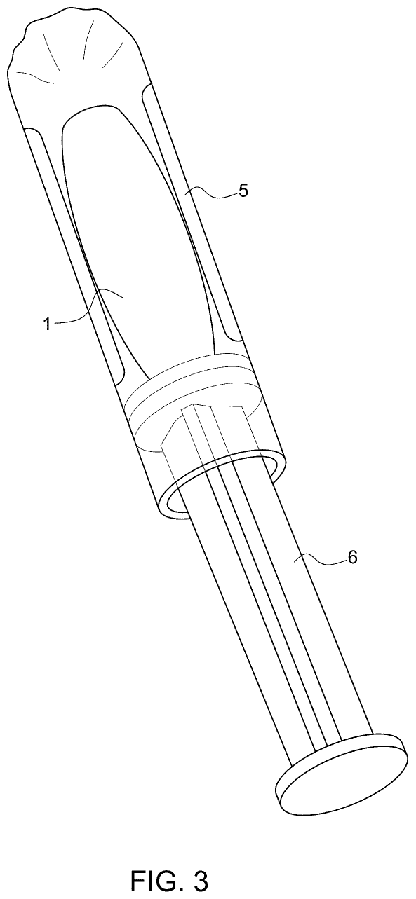

As illustrated in FIG. 3, prior to insertion, the device (1) is rolled up or otherwise appropriately configured and placed in the plastic base (5) of the applicator. The plunger (6) is, at this time, pulled back. The end of the plastic base furthest from the plunger is made using a soft silicone, and the patient or medical practitioner inserting the ostomy device may use a sterile lubricant for insertion of the ostomy device.

To insert the ostomy device, the device must first be placed in the applicator. Then the sterile lubricant is applied; the plastic base with the silicone tip is inserted into the stoma, and the plunger is used to push the end intended for insertion into the opening of the stoma that is expelling effluent. The plastic base and device are of sufficient total length that only the portion intended to be inside the body is pushed into the body. The plastic base and plunger are then removed, revealing the balloon port or ports (4c). The appropriate port or ports are used to fill both of the first balloon within the body and, if applicable, the second balloon outside of the body aiding in directing effluent into the pouch or bag.

The plastic base and plunger may be disposable and come with the ostomy device to prevent concerns regarding germ exposure or transmission, or may be of a suitable material and design for regular sterilization via known, medically accepted technology, such as steam sterilization, for re-use. Although the base is referred to as a plastic base, it may may be made of any suitable material, including but not limited to a sterile disposable cardboard or thin plastic, silicone, non-disposable plastic, or stainless steel. Because correct packing of the device within the plastic base will determine whether it inserts into the body without warping, tearing, or inadvertent irritation of the stoma from rough movement when the plunger is pushed, the ostomy device may be provided from the manufacturer in sterile packaging, with collection for sterilization and reuse as an option for the end user. Ideally, the dimensions of the balloon as inflated, balloon wall thickness, and channel thickness will be such that the balloon or intended to be inflated inside the body will be rolled up inside the channel and the plunger will push it out without difficulty or hazard either in the insertion into the body or in the removal of the plastic base and plunger. The balloon or balloons are ring shaped to help maintain the openness and positioning of the channel.

Because different patients have different dimensions, the device will be available in different lengths or sizes to enable the best fit. This is especially useful for patients with retracted stomas, by enabling effluent or discharge to exit the stoma and be directed into the stoma pouch as intended instead of pooling and causing damage to the skin surrounding the stoma and or to the seals on the stoma pouch or bag. There may also be different diameters of the inside ring in the possible case of a parastomal hernia, where the opening in the fascial layer may be wider than others.

While illustrative embodiments of the invention have been shown and described, variations and alternative embodiments will occur to those skilled in the art. Such variations and alternative embodiments can be made without departing from the scope of the invention as defined in the claims.

As used in this specification and the appended claims, the singular forms “a” and “an” indicate a single element, while “the” may refer back to single or plural referents. Unless defined otherwise, all technical and scientific terms used herein have the same meaning as commonly understood by one of ordinary skill in the art to which the disclosure pertains.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. No limitation is intended by the description of exemplary embodiments which may have included tolerances, feature dimensions, specific operating conditions, engineering specifications, or the like, and which may vary between implementations or with changes to the state of the art, and no such limitation should be implied therefrom. Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112 (f), unless the element is expressly recited using the exact phrase “means for . . . ” and no method or process step herein is to be construed under the provisions of 35 U.S.C. section 112 (f) unless the step, or steps, are expressly recited using the exact phrase “step(s) for . . . ”.

1. An insertable anti-leaking ostomy device, comprising: a tube having a first end and a second end, a first inflatable balloon, and a first balloon valve, wherein the first inflatable balloon is positioned on the first end and the first end and first inflatable balloon are configured to be inserted through the stoma into a patient via an applicator.

2. The insertable anti-leaking ostomy device of claim 1, wherein the first inflatable balloon is shaped like a ring, such that inflation of the first inflatable balloon will result in the balloon holding open the first end of the tube, and the device is configured for inflation of the first inflatable balloon with sterile water or saline solution via the first balloon valve after insertion into the patient.

3. The insertable anti-leaking ostomy device of claim 1, further comprising a second inflatable balloon shaped like a ring, wherein the second inflatable balloon is positioned on the second end of the tube.

4. A method of inserting the insertable anti-leaking ostomy device of claim 1, comprising:
first, rolling the first inflatable balloon and first end of the tube inward;
second, inserting the insertable anti-leaking ostomy device into a base of an applicator with the second end of the insertable anti-leaking ostomy device nearest a plunger of the applicator;
third, inserting the applicator which contains the insertable anti-leaking ostomy device through the stoma;
fourth, pressing the plunger of the applicator to push the first end and first inflatable balloon of the insertable anti-leaking ostomy device into the stoma through which the ostomy drains;
fifth, removing the entire applicator while leaving the first end and first inflatable balloon of the insertable anti-leaking ostomy device in the stoma;
sixth, using the balloon port of the inflatable anti-leaking ostomy device to inflate the first balloon of the inflatable anti-leaking ostomy device by filling it with sterile water or saline solution; and
seventh, directing the second end of the inflatable anti-leaking ostomy device into the ostomy pouch such that the ostomy drains through the inflatable anti-leaking ostomy device into the ostomy pouch.

5. A method of inserting the insertable anti-leaking ostomy device of claim 3, comprising:
first, rolling the first inflatable balloon and first end of the tube inward;
second, inserting the insertable anti-leaking ostomy device into a base of an applicator with the second end of the insertable anti-leaking ostomy device nearest a plunger of the applicator;
third, inserting the applicator which contains the insertable anti-leaking ostomy device through the stoma;
fourth, pressing the plunger of the applicator to push the first end and first inflatable balloon of the insertable anti-leaking ostomy device into the cavity which the stoma drains;
fifth, removing the entire applicator while leaving the first end and first inflatable balloon of the insertable anti-leaking ostomy device in the cavity which the stoma drains;
sixth, using the balloon port of the inflatable anti-leaking ostomy device to inflate the first balloon of the inflatable anti-leaking ostomy device by filling it with sterile water or saline solution;
seventh, inserting the second balloon into the an ostomy pouch; and
eighth, inflating the second balloon by inserting sterile water or saline solution via the second balloon port.
